(19) United States
(12) Patent Application Publication
(10) Pub. No.: US 2014/0248396 A1
(43) Pub. Date: Sep. 4, 2014

(54) Improved Viability of Probiotic Microorganisms Using Poly - gamm- Glutamic Acid

(71) Applicants: Iza Radecka, West Midlands (GB); David Hill, Wolverhampton (GB); Terence Bartlett, Wolverhampton (GB); Aditya Bhat, Aberystwyth (GB); Gopal Kedia, Newport (GB)

(72) Inventors: Iza Radecka, West Midlands (GB); David Hill, Wolverhampton (GB); Terence Bartlett, Wolverhampton (GB); Aditya Bhat, Aberystwyth (GB); Gopal Kedia, Newport (GB)

(73) Assignee: University of Wolverhampton, Wolverhampton, West Midlands (GB)

(21) Appl. No.: 14/342,188

(22) PCT Filed: Aug. 31, 2012
(86) PCT No.: PCT/GB2012/052149

§ 371 Date: Apr. 16, 2014

(30) Foreign Application Priority Data
Sep. 2, 2011 (GB) 1115143.8

(51) Int. Cl. A23L 1/30; A23C 9/12; C12N 1/20

(52) U.S. Cl. CPC A23L 1/3014; C12N 1/20; A23C 9/1203
USPC 426/61; 435/252.1; 435/71.2

(57) ABSTRACT

The invention relates to a probiotic microorganism at least partially coated with a poly glutamic acid, for example poly-γ-glutamic acid (γ-PGA). The invention also relates to an ingestible product comprising the probiotic microorganism at least partially coated with a poly glutamic acid and a method of making a probiotic microorganism at least partially coated with a poly glutamic acid. The invention also relates to a method of making poly-γ-glutamic acid (γ-PGA).

The present invention relates to the use of poly-γ-glutamic acid (γ-PGA) in improving the viability of probiotic microorganisms. The invention also relates to methods of manufacturing γ-PGA, in particular γ-PGA with specific properties.

Probiotics were defined by the World Health Organisation in 2002 as live microorganisms that when administered in adequate amounts confer a health benefit on the host. Accordingly probiotic microorganisms have been introduced into a variety of food and drink products for administration to a human or animal. However, many of the probiotic microorganisms used in food and drink products do not survive for long enough to confer a health benefit on the host. The processes that they must be subjected to to get them into the food or drink product and the human or animal body result in many becoming unviable before they have the chance to affect the host in any way.

Firstly the probiotic microorganisms are subjected to a freeze drying process. Secondly the probiotic microorganisms are incorporated into a food or drink product and then stored for a period of time that depends on the product they have been incorporated into. Finally the probiotic microorganisms are ingested by a human or animal with the food or drink product; the conditions of the mouth and stomach in particular being adverse to the viability of the probiotic microorganisms. Accordingly having passed through all of the above processes there are only relatively few microorganisms remaining viable and able to exert some effect on the host.

Furthermore the nature of the food or drink product into which the probiotic microorganism is introduced is generally limited to milk based products which maintain a weak acid environment tolerated by the microorganisms. However, even under these conditions, shelf life is restricted.

Therefore, there remains a need for a means to improve the viability of probiotic microorganisms as they pass through, in particular, the freeze drying, storage and ingestion processes. It would also be useful to be able to put the microorganisms into food and drink products that are not milk based.

Accordingly the present invention provides an ingestible product comprising a probiotic microorganism and a poly glutamic acid.

The poly glutamic acid is preferably poly-γ-glutamic acid (γ-PGA).

The γ-PGA preferably has a molecular weight of from 10,000 to 1,000,000 Daltons (Da). The γ-PGA may have a molecular weight of from 20,000 to 800,000 Da or 20,000 to 700,000 Da or 20,000 to 600,000 Da. More preferably the γ-PGA may have a molecular weight of from 50,000 to 500,000 Da, for example 100,000 to 300,000 Da or 200,000 to 260,000 Da.

The γ-PGA may be produced in any suitable bacteria, for example Bacillus subtilis and Bacillus licheniformis. In one embodiment the γ-PGA is produced in Bacillus subtilis, for example Bacillus subtilis natto.

The probiotic microorganism may be a bacteria. Examples of possible bacteria include Bifidobacterium longum, Bifidobacterium breve and Lactobacillus casei.

All or part, of some or all, of the probiotic microorganisms may be coated with biopolymer, for example by suspending the probiotic microorganisms in biopolymer with subsequent freeze drying.

All or part, of some or all, of the probiotic microorganisms may be coated with the poly glutamic acid, for example by suspending the probiotic microorganisms in the polyglutamic acid with subsequent freeze drying

The ingestible product may be a food or beverage product. The ingestible product may be milk based, for example yogurt or a yogurt drink, or non-milk based, for example fruit based such as a fruit juice.

Also provided is a probiotic microorganism at least partially coated with a poly glutamic acid.

The poly glutamic acid is preferably poly-γ-glutamic acid (γ-PGA).

The γ-PGA preferably has a molecular weight of from 10,000 to 1,000,000 Daltons (Da). The γ-PGA may have a molecular weight of from 20,000 to 800,000 Da or 20,000 to 700,000 Da or 20,000 to 600,000 Da. More preferably the γ-PGA may have a molecular weight of from 50,000 to 500,000 Da, for example 100,000 to 300,000 Da or 200,000 to 260,000 Da.

The γ-PGA may be produced in any suitable bacteria, for example Bacillus subtilis and Bacillus licheniformis. In one embodiment the γ-PGA is produced in Bacillus subtilis, for example Bacillus subtilis natto.

The probiotic microorganism may be a bacteria. Examples of possible bacteria include Bifidobacterium longum, Bifidobacterium breve and Lactobacillus casei.

Also provided is method of at least partially coating a probiotic microorganism with a poly glutamic acid comprising mixing the microorganism with a solution of a poly glutamic acid.

The poly glutamic acid may be provided in a concentration of from 2 to 15% (w/v), for example from 5 to 12% (w/v), such as 10% (w/v).

The microorganism may be provided in cell pellet form.

The probiotic microorganism may be a bacteria. Examples of possible bacteria include Bifidobacterium longum, Bifidobacterium breve and Lactobacillus casei.

The poly glutamic acid is preferably poly-γ-glutamic acid (γ-PGA).

The γ-PGA preferably has a molecular weight of from 10,000 to 1,000,000 Daltons (Da). The γ-PGA may have a molecular weight of from 20,000 to 800,000 Da or 20,000 to 700,000 Da or 20,000 to 600,000 Da. More preferably the γ-PGA may have a molecular weight of from 50,000 to 500,000 Da, for example 100,000 to 300,000 Da or 200,000 to 260,000 Da.

The γ-PGA may be produced in any suitable bacteria, for example Bacillus subtilis and Bacillus licheniformis. In one embodiment the γ-PGA is produced in Bacillus subtilis, for example Bacillus subtilis natto.

Also provided is a method of making poly-γ-glutamic acid (γ-PGA) comprising the steps of:

a) preparing a starter culture of the appropriate bacterial colony in fermentation broth;

b) adding starter culture to a growth medium;

c) producing γ-PGA in the growth medium.

In the context of this invention the “starter culture” comprises one or more colonies of an appropriate strain of bacteria, preferably one or more highly mucoid colonies, inoculated in a growth medium, for example, TSB and incubated, for example at 37° C. for 24 h.

The bacterial colony may be B. subtilis or B. licheniformis. The bacterial colony is preferably B. subtilis natto.

The fermentation broth may be any suitable broth, for example tryptone soy broth (TSB).

The growth media may be any suitable growth media, for example growth media E or growth media GS.

Growth medium GS generally produces a higher yield of γ-PGA than growth medium E. The growth media can affect the crystallinity of the γ-PGA with the γ-PGA produced in medium E being amorphous and the γ-PGA produced in medium GS being crystalline. Furthermore the growth medium has an effect on the formation of the salt or free acid form of the γ-PGA. In growth medium GS most of the γ-PGA produced was the sodium salt whereas in growth medium E a considerable amount of the acid form was produced. Molecular weight is affected by both growth medium and strain of bacteria.

The bacterial colony may be highly mucoid.

The fermentation step may take place at a temperature of 35 to 39° C., for example 37° C.

The fermentation time may be from 18 to 30 hours, for example from 20 to 28 hours, such as 24 hours.

The growth step may take place at a temperature of 35 to 39° C., for example 37° C.

The growth time may be from 90 to 100 hours, for example from 94 to 98 hours, such as 94 hours.

The growth step may include the steps of agitating in any suitable fermenter vessel for all or part of the growth time.

Agitation can range from 100 rpm to 1000 rpm.

Any suitable fermenter vessel could be used including, for example, shake flasks, aerated stirred tank reactors and solid state fermenters.

The poly-γ-glutamic acid may be isolated from the growth media. The poly-γ-glutamic acid in the growth media may first be subjected to centrifugation.

Any suitable alcohol based solvent, such as ethanol, may then be added to the cell free supernatant resulting from centrifugation, for example at a ratio of 2:1 to 6:1, for example 4:1, alcohol to supernatant. The alcohol/supernatant mixture may be incubated at 2 to 6° C., for example 4° C. for 70 to 75 hours, for example 72 hours.

The poly-γ-glutamic acid may be removed from the alcohol/supernatant mixture by centrifugation and/or filtration.

The poly-γ-glutamic acid may be subjected to lyophilization. The poly-γ-glutamic acid may be frozen before being subjected to lyophilization

Aspects of the invention will now be described by reference to experimental work carried out. The experimental work is purely illustrative of the aspects of the invention and is in no way intended to be limiting on the scope of the invention.

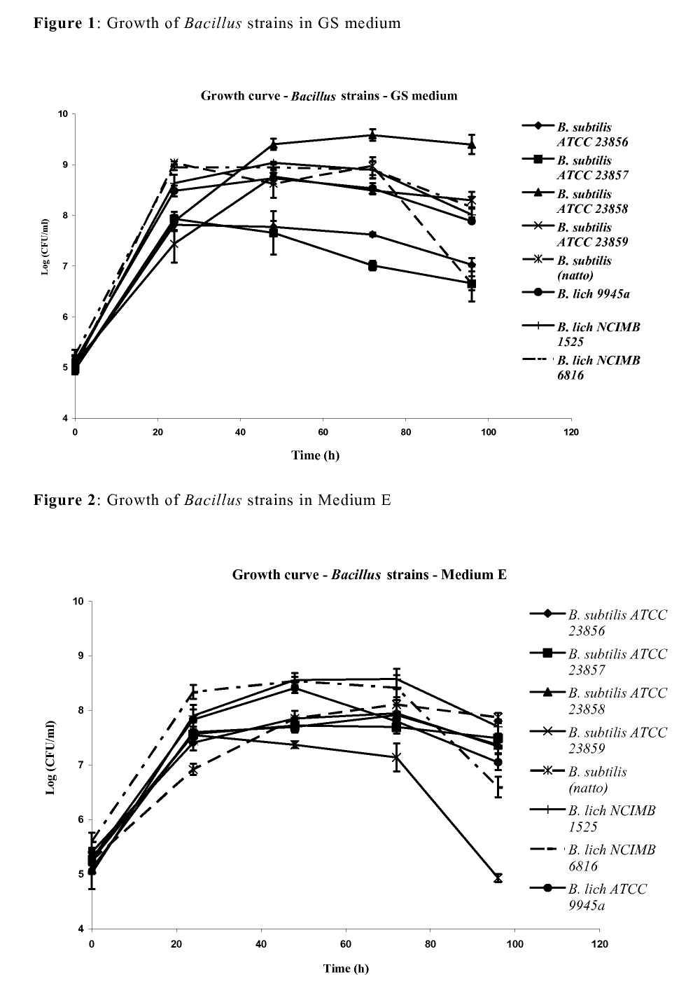

FIG. 1 shows the growth of the Bacillus strains in GS medium;

FIG. 2 shows the growth of the Bacillus strains in E medium;

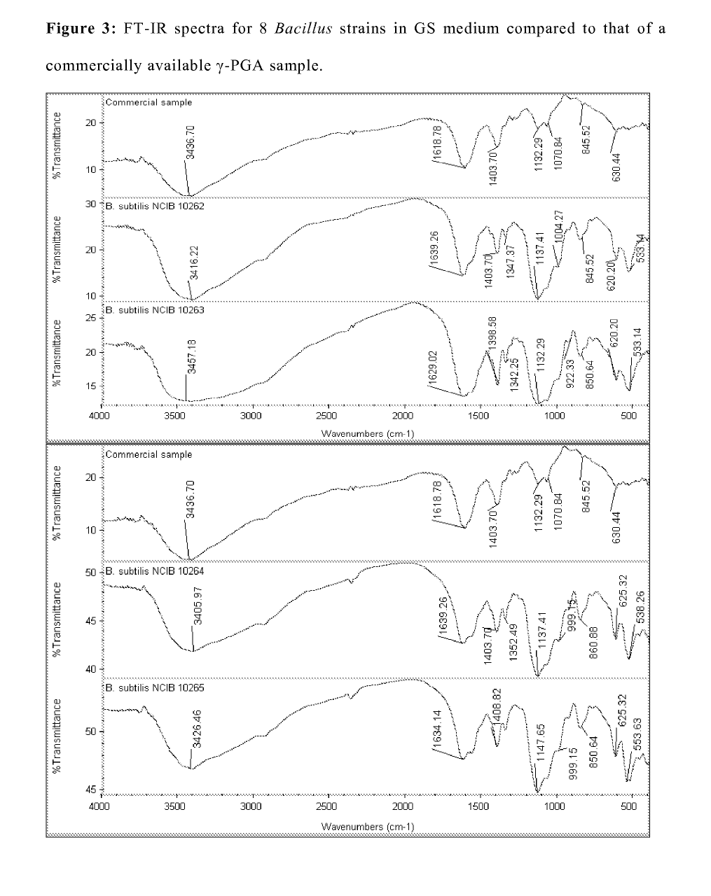

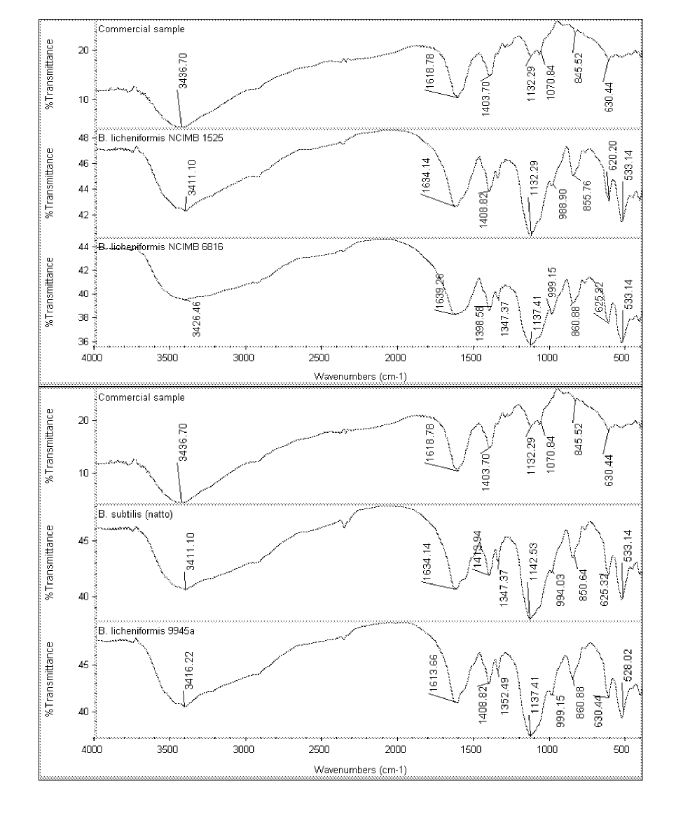

FIG. 3 shows FT-IR spectra for the γ-PGA produced by the Bacillus strains compared to that of a commercially available γ-PGA sample;

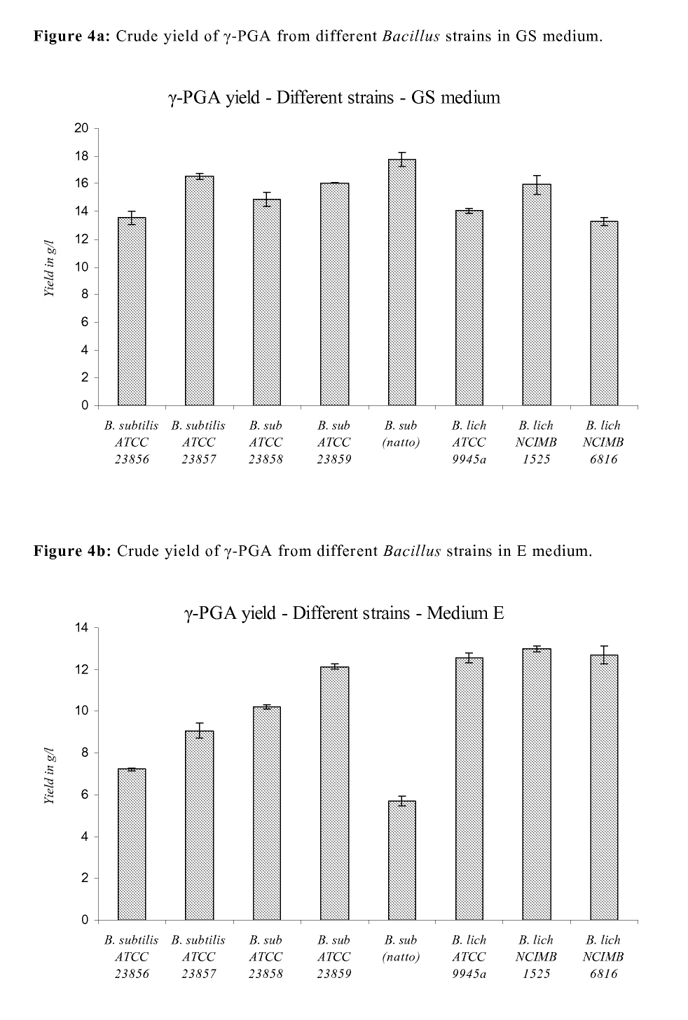

FIG. 4 shows crude yield of γ-PGA from different Bacillus strains in growth media E and GS;

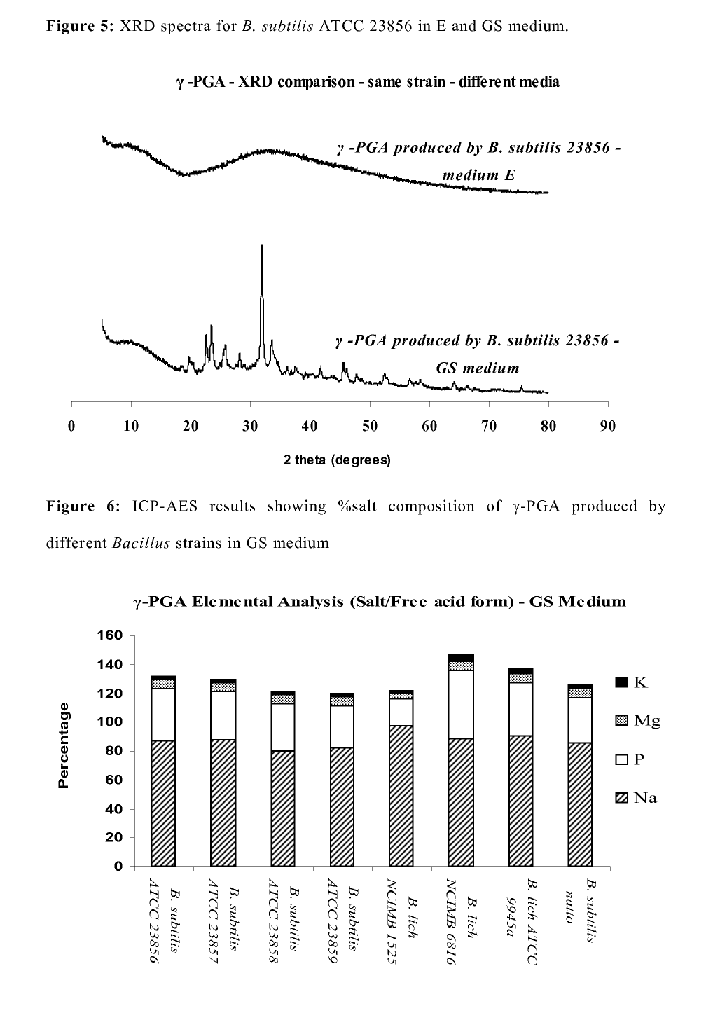

FIG. 5 shows the XRD spectra for the strain—B. subtilis ATCC 23856 in GS and E medium;

FIG. 6 shows ICP-AES results showing % salt composition of γ-PGA produced by the different Bacillus strains in GS medium;

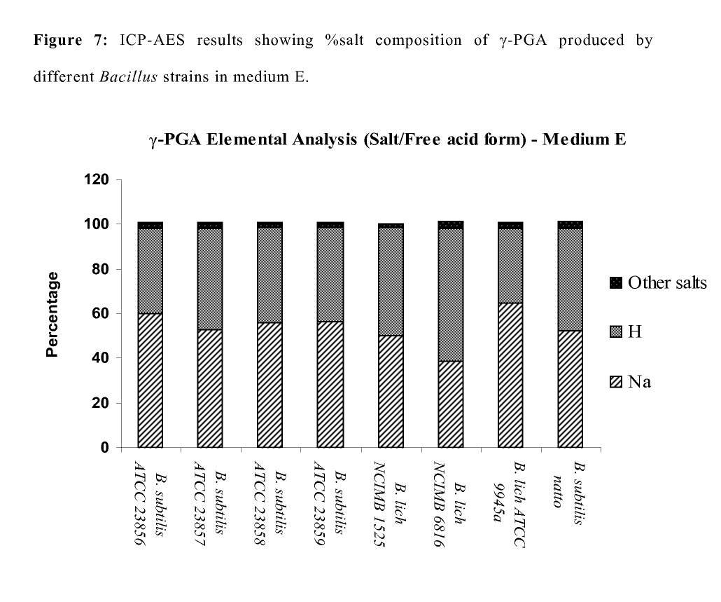

FIG. 7 shows ICP-AES results showing % salt composition of γ-PGA produced by the different Bacillus strains in E medium;

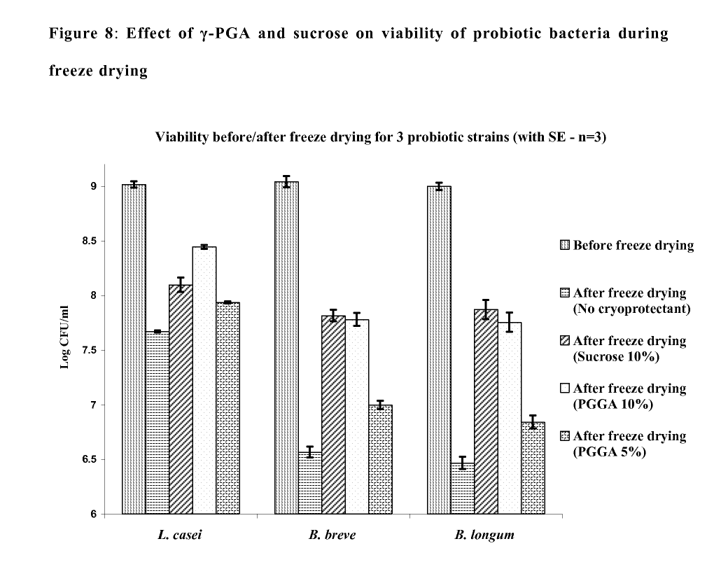

FIG. 8 shows the effect of pressure cooked γ-PGA and sucrose on viability of probiotic bacteria during freeze drying;

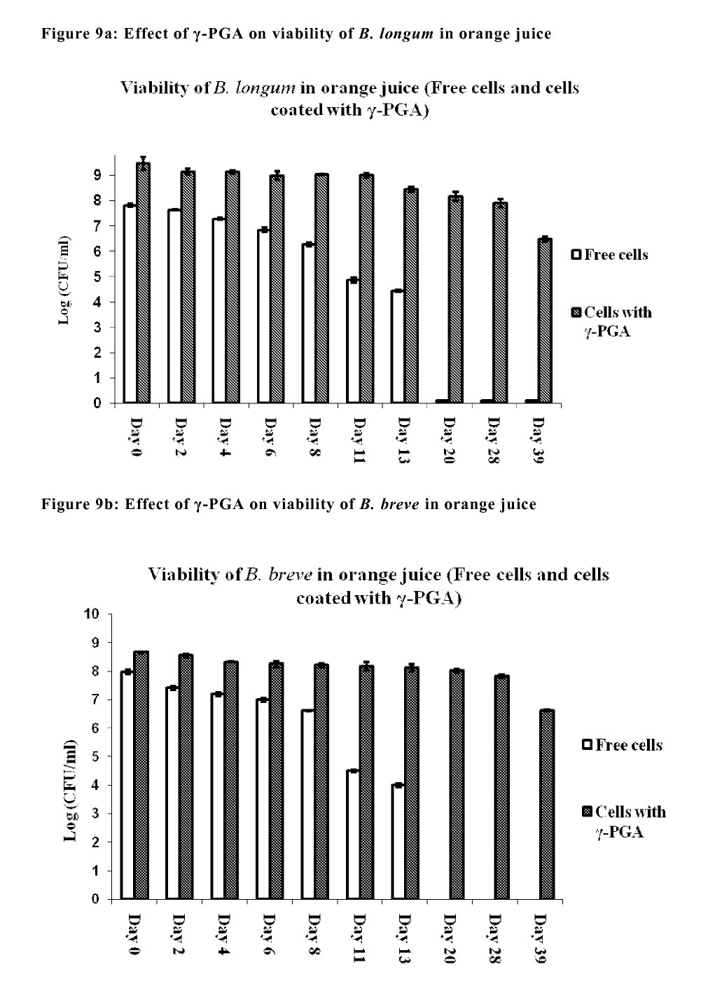

FIGS. 9a and b show the effect of γ-PGA on the viability of B. longum and B. breve in orange juice; and

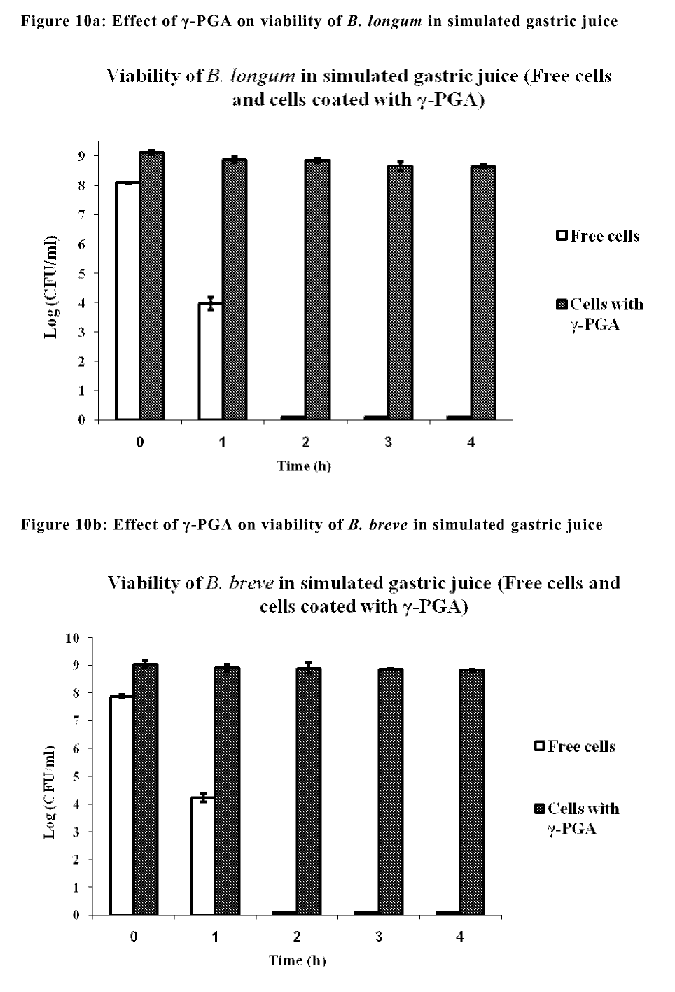

FIGS. 10a and b show the effect of γ-PGA on the viability of B. longum and B. breve in simulated gastric juice.

### PART 1. PRODUCTION OF POLY-γ-GLUTAMIC ACID FROM EIGHT DIFFERENT STRAINS OF BACILLUS

This experiment describes the production of γ-PGA with 8 different strains of bacteria—B. subtilis natto, B. subtilis ATCC 23856, B. subtilis ATCC 23857, B. subtilis ATCC 23858, B. subtilis ATCC 23859, B. licheniformis 9945a, B. licheniformis NCIMB 1525 and B. licheniformis NCIMB 6816 in shake flasks.

1. Introduction

It is known from previous research that Bacillus subtilis and Bacillus licheniformis are able to produce extracellular γ-PGA which can be easily recovered from the production medium. In this work, five strains of Bacillus subtilis—B. subtilis natto, B. subtilis subsp. subtilis ATCC 23856 (also known as B. subtilis subsp. subtilis SB19 EMG50), B. subtilis ATCC 23857 (also known as B. subtilis subsp. subtilis 168 EMG51), B. subtilis ATCC 23858 (also known as B. subtilis subsp. subtilis EMG52), B. subtilis ATCC 23859 (also known as B. subtilis subsp. subtilis EMG53) and three strains of Bacillus licheniformis—B. licheniformis 9945a, B. licheniformis NCIMB 1525 (also known as B. licheniformis 1229) and B. licheniformis NCIMB 6816 (also known as B. licheniformis Glaxo417) were investigated for the production of γ-PGA.

To try to obtain γ-PGA with different properties, such as crystallinity, form of peptide and yield along with molecular weight, two different media were used for production.

2. Material and Methods

2.1 Bacterial Strains

All strains—B. subtilis natto, B. subtilis ATCC 23856, B. subtilis ATCC 23857, B. subtilis ATCC 23858, B. subtilis ATCC 23859, B. licheniformis 9945a, B. licheniformis NCIMB 1525 and B. licheniformis NCIMB 6816—were obtained from National Collection of Industrial and Marine Bacteria (NCIMB). The stock cultures were freeze-dried and stored at −20° C. Before use, cultures were revived aseptically and grown on general purpose microbiology media overnight at 37° C.

2.2 Growth Media

Tryptone soya agar (TSA), tryptone soya broth (TSB) and one-quarter strength ringer solution were prepared according to the manufacturer's protocol (Lab M, UK).

The composition of GS medium and Medium E has been given in Table 1 & 2 below. The pH of both media was adjusted to 7.2 using 3 M NaOH and 1 M HCl.

All media were prepared using de-ionised water and sterilized at 121° C. at 15 p.s.i. for 20 min. Sucrose solution was sterilised separately in a pressure cooker (110° C. at 5 p.s.i. for 30 min) and vitamin solution was filter sterilised (0.2 μm Ministart) and added separately to GS medium.

TABLE 1
Composition of GS medium

| Chemical | Amount | | Source |
|---|---|---|---|
| L-glutamic acid | 20 | g/L | Sigma-Aldrich |
| Sucrose | 50 | g/L | Acros Organics |
| Potassium dihydrogen orthophosphate, | 2.7 | g/L | Fisher Scientific |
| KH₂PO₄ | | | |
| Di-sodium hydrogen phosphate | 4.2 | g/L | AnalaR |
| anhydrous, Na₂HPO₄ | | | |
| Sodium chloride, NaCl | 50 | g/L | Aldrich Chemical |
| | | | Co. Ltd |
| Magnesium sulphate heptandrate, | 5 | g/L | AnalaR |
| MgSO₄•7H₂0 | | | |
| Murashige-Skoog vitamin solution | 1 | ml/L | Sigma-Aldrich |

TABLE 2
Composition of Medium E

| Chemical | Amount | Source |
|---|---|---|
| L-glutamic acid | 20 g/L | Sigma-Aldrich ® |
| Citric acid | 12 g/L | Acros Organics |
| Glycerol | 80 g/L | Avocado |
| Ammonium chloride, NH₄Cl | 7 g/L | Riedel-de Haen |
| Magnesium sulphate heptandrate, | 0.5 g/L | AnalaR |
| MgSO₄•7H₂0 | | |
| Iron (III) chloride hexahydrate, | 0.2 g/L | Sigma-Aldrich ® |
| FeCl₃•6H₂0 | | |
| Di-potassium hydrogen orthophosphate | 0.5 g/L | AnalaR (BDH) |
| anhydrous, K₂HPO₄ | | |
| Calcium chloride dehydrate, CaCl₂•2H₂0 | 0.15 g/L | Fisher Chemicals |
| Manganese (II) sulphate monohydrate, | 0.2 g/L | Sigma |
| MnSO₄•H₂0 | | |

2.3 Production of γ-PGA

Production of γ-PGA with each bacterial strain was done in triplicates. Highly mucoid colonies of the appropriate strain were selected and inoculated in flasks containing 250 ml TSB and incubated at 37° C. for 24 h. At the end of 24 h, the starter culture reached a population of ˜7 log CFU/ml. 5% of this culture was inoculated into 250 ml of production medium—GS and E. All flasks were incubated at 37° C. on a rotary shaker (Innova 43) at 150 rpm for 96 h. Samples were taken at 0, 24, 48, 72 and 96 h for analysis of cell growth and nutrient consumption.

2.4 Isolation of γ-PGA

After 96 h, the cell suspension was centrifuged at 17000 g for 30 minutes (Hermele 2 300K). Four volumes of cold 90% (vv) ethanol was added to the cell free supernatant and incubated at 4° C. for 72 h. Wet γ-PGA powder was obtained as sediment. The sediment was separated from the supernatant by centrifugation at 17000 g for 30 mins. The obtained polymer was prepared for lyophilisation by dissolving it in 10 ml of deionised water in round bottom flasks. The flasks were then rotated gently on a mixture of 90% (vv) ethanol at −20° C. and dry ice to freeze the biopolymer in the form of a thin film. The frozen biopolymer was then lyophilized to obtain dry γ-PGA powder (Edward Modulo). The dried powder was weighed to calculate yield in g/l and stored in a desiccator for further analysis.

2.5 Determination of Growth

To monitor cell growth, samples were taken aseptically at 0, 24, 48, 72 and 96 h to monitor cell growth. Miles and Misra technique was used to calculate Colony Forming Units/ml (CFU/ml) in triplicates. Serial dilutions from 10-1 to 10-10 were performed by dispensing 0.5 ml of sample in 4.5 ml of sterile ¼th strength ringer solution. 20 μl of each dilution was dispensed onto TSA plates under aseptic conditions. The plates were then incubated at 37° C. for 24 h after which plates with less than 20 colonies were selected and used for viable count determination. CFU/ml was calculated with the formula:

$$\frac{CFU}{ml} = n \times \frac{1}{( Sample volume )} \times \frac{1}{D . F .}$$

: where n is the no. of colonies and D.F. is dilution factor.

2.6 Chemical Analysis

Identification of γ-PGA

Isolated biopolymer was analyzed using Fourier Transformed Infra Red spectroscopy (FTIR) with an Impact 404 Nicolet spectrometer (UK) with KBr pellet in conjunction with OMNIC software. The FTIR spectra of the produced γ-PGA were compared with the spectra of a commercially available γ-PGA sample.

Elemental Analysis

To evaluate if isolated polymer was in the form of free acid or salt, elemental analysis was performed using Inductively Coupled Plasma Atomic Emission Spectroscopy (ICP-AES) with SPECTRO CIROScco. Prior to the analysis, 6 ml HNO3 (70%) and 1 ml H2O2 (30% wv) was added to 0.25 g of dried γ-PGA and this solution was digested with ETHOS 900 Microwave Labstation—Milestone Microwave Laboratory System.

Molecular Weight

Aqueous based gel permeation chromatography (GPC) was used to determine molecular weight (Mw), molecular number (Mn) and polydispersity (Smithers Rapra) using a MZ Hema guard plus 2× Hema Linear column. 0.2 M NaNO3, 0.01 M NaH2PO4 at pH 7 was used as the eluent with a flow rate of 1.0 ml/min at 30° C. and an RI detector. The data was collected and analyzed using Polymer Laboratories “Cirrus” software. GPC system used for this work was calibrated with sodium polyacrylate calibrants obtained from Polymer Laboratories.

Nutrient Consumption Analysis

Nutrient consumption analysis was performed using High Performance Liquid Spectroscopy (HPLC) with an HP series 1100 HPLC machine at the University of Reading, U.K. A Prevail Organic Acid 5u column with a UV detector was used for analyzing L-glutamic acid and citric acid whereas a Phenomenex carbohydrate column (Rezex RCM—monosaccharide Ca++—8%) was used for sucrose and glycerol determination. Filtered deionised water was used as the eluent for sucrose analysis. For organic acid analysis, 25 mM KH2PO4 (pH 2.5 adjusted with phosphoric acid) was used as the eluent. Before loading, the samples were filtered using 0.45 μm filters and were diluted 10 fold.

Crystallinity

Crystallinity was assessed using powder X-Ray Diffraction (XRD) analysis. Data was collected at room temperature with a Phillips PW1700, 40 kV/40 mA, CuKα instrument.

Statistical Analyses

All results were analyzed using Microsoft Excel 2003. Two way Annova was used to compare data in Microsoft Excel 2003.

3. Results and Discussion

3.1 Growth

FIGS. 1 and 2 show the growth of all bacterial strains in both GS and E medium. At 0 h, all the strains had a cell count of ˜5-6 CFU/ml. In GS medium, B. subtilis 23858 reached a maximum cell count of log 9.58 at 72 h. In medium E, B. licheniformis 1525 reached a maximum cell count of log 8.57 at 72 h. The maximum cell counts for the B. subtilis strains (˜log 7.5-8.1 CFU/ml) did not reach as high as that of the B. licheniformis strains (˜log 8.5 CFU/ml) in medium E. B. subtilis 23858 & 23859 and B. licheniformis 9945a, 1525 & 6816 had a higher cell count at 96 h in GS medium than in Medium E. B. subtilis 23859 did not grow well in medium E with a cell count of ˜log 4.9 CFU/ml at the end of 96 h.

3.2 Nutrient Consumption

Tables 3 and 4 below show utilization of nutrients by bacterial strains in both GS and E media after 96 h. In GS medium, except B. subtilis 23859 (75.71%), all strains utilized more than 85% of provided sucrose at the end of 96 h. In contrast, the C source in Medium E, i.e. glycerol, was not consumed as much (49.56-70.67%). In GS medium, B. subtilis strains consumed more L-glutamic acid than B. licheniformis strains. B. subtilis natto consumed the most L-glutamic acid at the end of 96 h (˜95%). Incidentally, B. subtilis natto also produced the highest yield of γ-PGA in GS medium (˜17.7 g/l).

In contrast, in medium E, B. licheniformis strains consumed more L-glutamic acid than B. subtilis strains. This could possibly be reflected in the fact that the B. licheniformis strains produced a slightly higher yield of γ-PGA in medium E than most of the B. subtilis strains at the end of 96 h.

Since all the experiments were done in shake flasks, the pH and aeration of the culture could not be controlled which could have some impact on nutrient consumption.

TABLE 3
Nutrient utilization in GS medium after 96 h

| Strain | B. subtilis | B. subtilis | B. subtilis | B. subtilis | B. subtilis | B. licheniformis | B. licheniformis | B. licheniformis |
|---|---|---|---|---|---|---|---|---|
| Name | 23856 | 23857 | 23858 | 23859 | natto | 1525 | 6816 | 9945a |
| % | 90.66 | 88.19 | 90.77 | 75.71 | 89.97 | 94.77 | 97.96 | 86.98 |
| decrease | | | | | | | | |
| in | | | | | | | | |
| Sucrose | | | | | | | | |
| % | 64.72 | 58.48 | 82.26 | 67.25 | 95.01 | 39.22 | 33.84 | 17.46 |
| decrease | | | | | | | | |
| in L- | | | | | | | | |
| glutamic | | | | | | | | |
| acid | | | | | | | | |

TABLE 4
Nutrient utilization in Medium E after 96 h.

| Strain | B. subtilis | B. subtilis | B. subtilis | B. subtilis | B. subtilis | B. licheniformis | B. licheniformis | B. licheniformis |
|---|---|---|---|---|---|---|---|---|
| Name | 23856 | 23857 | 23858 | 23859 | natto | 1525 | 6816 | 9945a |
| % | 49.89 | 54.18 | 50.25 | 62.97 | 49.56 | 65.64 | 67.58 | 70.67 |
| decrease | | | | | | | | |
| in | | | | | | | | |
| Glycerol | | | | | | | | |
| % | 24.44 | 29.72 | 28.50 | 26.3 | 23.30 | 34.22 | 44.46 | 36.03 |
| decrease | | | | | | | | |
| in L- | | | | | | | | |
| glutamic | | | | | | | | |
| acid | | | | | | | | |

3.3 Identification

After isolation of γ-PGA, it was seen that cells grown in GS medium produced a white powder. In medium E, cells produced a brownish product, possibly due to the presence of ferric ions. The γ-PGA produced by all strains was identified by IR. All the strains showed similar results and the FT-IR spectra for γ-PGA obtained by the strains under investigation compared well to that of a commercially available γ-PGA sample (see FIG. 3).

3.4 Yield

All strains produced more γ-PGA in GS medium than that in medium E, except in the case of B. licheniformis 9945a as shown in FIG. 4. For B. licheniformis 9945a, the yields in both media are comparable. B. subtilis natto produced the maximum yield in GS medium (˜17.7 g/l). This strain utilized more L-glutamic acid than any other strain in our study (˜95%) and this could probably be reflected in the yield. However, the yields of the other strains do not necessarily correlate to the amount of L-glutamic acid consumed. Hence, the yield of γ-PGA could not only be dependent on the consumption of exogenous L-glutamic acid, but also on the ability of the bacteria to produce endogenous L-glutamic acid for production of γ-PGA. Medium E is known to be most suitable for production of γ-PGA with B. licheniformis 9945a, which is probably the most studied strain for the production of γ-PGA. All B. licheniformis strains produced more γ-PGA in medium E when compared to the B. subtilis strains, except B. subtilis 23859. B. subtilis 23859 consumed more glycerol in medium E than the other B. subtilis strains and this could be the reason why its yield of γ-PGA was slightly better. Since nutrient consumption could be affected by pH of the medium, the yield could also be affected.

3.5 Crystallinity

Amorphous γ-PGA is easily soluble in water, whereas a crystalline form of γ-PGA is relatively insoluble in water. XRD analysis showed that all strains produced amorphous γ-PGA with medium E. In contrast, a crystalline powder was obtained when the cells were grown in GS medium, which was evident because of the presence of distinct peaks on the spectra. The XRD spectra for one such strain—B. subtilis ATCC 23856 in GS and E medium has been shown in FIG. 5.

3.6 Elemental Analysis

Elemental analysis was performed to identify whether the salt or the acid form of γ-PGA was produced. ICP-AES analysis breaks down the crude polymer and measures the concentration of individual elements that make up the polymer. Hence impurities, if present, in the sample would also be detected. ICP-AES results showed that most of the γ-PGA obtained with cells grown in GS medium was in fact the sodium salt of γ-PGA (Na-γ-PGA) with some γ-PGA also in its P, Mg and K salt form, see FIG. 6. None of the strains produced the acid form of γ-PGA (H+-γ-PGA) in GS medium.

In contrast, strains grown in medium E produced considerable amount of H+-γ-PGA (37-57%) along with Na-γ-PGA, see FIG. 7. The pH of medium E was adjusted with the help of 3M NaOH.

The free acid form of γ-PGA is insoluble in water, whereas the salt forms of γ-PGA are fully soluble in water.

3.7 Molecular Weight

Molecular weight was determined with the help of aqueous based GPC. When grown in GS medium B. licheniformis NCIMB 1525 produced γ-PGA with a molecular weight of 871000 Da (polydispersity—1.35) while B. licheniformis NCIMB 6816 produced γ-PGA with a molecular weight of 850000 Da (polydispersity—1.45) and B. subtilis natto produced γ-PGA with a molecular weight of 257500 Da (polydispersity—4.75). The other strains produced a lower molecular weight product (˜3000 Da) With medium E, B. licheniformis NCIMB 6816 produced γ-PGA weighing 856500 Da (polydispersity—1.2) and B. licheniformis 9945a produced γ-PGA weighing 760000 Da (polydispersity—1.2) while the other strains produced a lower molecular weight product (˜<3000 Da).

A summary of the properties of γ-PGA obtained with the 8 Bacillus strains in both GS and E medium can be seen in Tables 5 and 6 below.

TABLE 5
Properties of γ-PGA produced from 8 Bacillus strains in GS medium

| | Yield | | | Molecular | |
|---|---|---|---|---|---|
| Organism | (g/l) | Crystallinity | % Na Salt | weight (Da) | Polydispersity |
| B. subtilis 23856 | 13.55 ± 0.78 | Crystalline | 86.90 | ~3000 | n/a |
| B. subtilis 23857 | 16.52 ± 0.35 | Crystalline | 87.55 | ~3000 | n/a |
| B. subtilis 23858 | 14.88 ± 0.84 | Crystalline | 80.26 | ~3000 | n/a |
| B. subtilis 23859 | 16.03 ± 0.06 | Crystalline | 82.40 | ~3000 | n/a |
| B. licheniformis | 15.93 ± 1.16 | Crystalline | 97.40 | 871000 | 1.35 |
| 1525 | | | | | |
| B. licheniformis | 13.3 ± 0.52 | Crystalline | 88.37 | 850000 | 1.45 |
| 6816 | | | | | |
| B. subtilis natto | 17.77 ± 0.9 | Crystalline | 85.38 | 257500 | 4.75 |
| B. licheniformis | 14.05 ± 0.31 | Crystalline | 90.30 | ~3000 | n/a |
| 9945a | | | | | |

TABLE 6
Properties of γ-PGA produced from 8 Bacillus strains in medium E.

| | Yield | | | % Acid | Molecular | |
|---|---|---|---|---|---|---|
| Organism | (g/l) | Crystallinity | % Na Salt | form | weight (Da) | Polydispersity |
| B. subtilis | 7.23 ± 0.07 | Amorphous | 59.90 | 37.76 | <3000 Da | n/a |
| 23856 | | | | | | |
| B. subtilis | 9.08 ± 0.34 | Amorphous | 52.68 | 44.98 | <3000 Da | n/a |
| 23857 | | | | | | |
| B. subtilis | 10.2 ± 0.09 | Amorphous | 56 | 41.91 | <3000 Da | n/a |
| 23858 | | | | | | |
| B. subtilis | 12.13 ± 0.12 | Amorphous | 56.29 | 41.73 | <3000 Da | n/a |
| 23859 | | | | | | |
| B. licheniformis | 12.98 ± 0.15 | Amorphous | 50.32 | 48.09 | <3000 Da | n/a |
| 1525 | | | | | | |
| B. licheniformis | 12.7 ± 0.45 | Amorphous | 38.84 | 57.97 | 856500 | 1.2 |
| 6816 | | | | | | |
| B. subtilis | 5.7 ± 0.25 | Amorphous | 52.31394 | 44.55611 | <3000 Da | n/a |
| natto | | | | | | |
| B. licheniformis | 12.58 ± 0.24 | Amorphous | 64.46865 | 32.88571 | 760000 Da | 1.2 |
| 9945a | | | | | | |

4. Conclusion

This study produced γ-PGA using 8 strains of Bacillus and the γ-PGA produced had different properties (high/low molecular weight, amorphous/crystalline, salt and acid form).

If the cells are grown in GS medium, they produce a crystalline salt form of γ-PGA. In contrast, when they are grown in Medium E, an amorphous acid form of γ-PGA is produced. Crystallinity and form of γ-PGA seem to be Bacillus strain independent and these properties could be manipulated with the medium of production.

Molecular weight however, depends on the strain and medium of production. It can be seen from the results that B. licheniformis 6816 produced a very high molecular weight γ-PGA in both Medium E and GS, but B. subtilis natto produced a high molecular weight product only in GS medium. B. licheniformis 9945a produced a high molecular weight polymer in Medium E, but not in GS medium.

### PART 2. SUMMARY OF TESTS AFTER INVESTIGATING PRODUCTION OF γ-PGA USING 8 STRAINS OF BACILLUS

1. Introduction

After producing γ-PGA using 8 different strains of Bacillus and characterizing its properties using various analytical techniques, Bacillus subtilis natto was chosen to produce γ-PGA for tests with probiotic bacteria.

2. Materials and Methods

To produce γ-PGA for the tests, batch cultures of B. subtilis natto were carried out in a 5 litre fermenter. The bacteria were first inoculated in 250 ml of tryptone soy broth for 24 h at 37° C. 5% of this inocolum was added to fermenter. The fermentation temperature was maintained at 37° C., and the pH of the culture was allowed to drop naturally without the addition of acid. Once pH 6.5 was attained, the pH was maintained by automated addition of 3 M NaOH or 3 M HCl. The stirring speed and airflow were 250 rpm and 1.0 l/min respectively at the start of fermentation. Since γ-PGA is an extracellular polymer, the culture medium becomes highly viscous with increasing polymer production. The increased viscosity decreases the volumetric oxygen mass transfer, leading to oxygen limitation. The supply of oxygen was maintained above 40% by controlling the agitation speed and air flow rate. After 96 h, the cell suspension was centrifuged at 17000 g for 30 minutes. Four volumes of cold 90% (vv) ethanol was added to the cell free supernatant and incubated at 4° C. for 72 h. Wet γ-PGA powder was obtained as sediment. The sediment was separated from the supernatant by centrifugation at 17000 g for 30 mins. The wet crude polymer was then dissolved in water and dialyzed to eliminate impurities lower than 10,000 Da. The obtained pure polymer was prepared for lyophilisation in round bottom flasks. The flasks were rotated gently on a mixture of 90% (v/v) ethanol at −20° C. and dry ice to freeze the biopolymer in the form of a thin film. The frozen biopolymer was then lyophilized to obtain dry γ-PGA powder. The pure dried powder was weighed to calculate yield in g/l and stored in a desiccator for tests with probiotics.

3. Tests with Probiotics

3.1 γ-PGA as a Cryoprotectant

Three probiotic bacteria, Bifidobacterium longum, Bifidobacterium breve and Lactobacillus casei were used for the tests. The effect of 10% Na-γ-PGA was tested on viability of the bacteria before and after freeze drying. To coat cells with cryoprotectant before freeze drying, Bifidobacteria were inoculated in TPY broth (22 h for B. breve and 16 h for B. longum) and Lactobacillus casei was inoculated in MRS broth (for 48 h) at 37° C. anaerobically. After incubation, viable counts were taken on TPY agar (Bifidobacteria) and MRS agar (Lactobacillus) to determine number of viable cells before freeze drying. The cultures were then centrifuged and washed with PBS to obtain cell pellets. Cells were then mixed thoroughly in 10 ml solutions of 10% Na-γ-PGA, 5% Na-γ-PGA and 10% sucrose. For cells without a cryoprotectant, 10 ml of water was added. Cells were then frozen at −80° C. for 24 h and freeze dried to obtain a dry powder. After freeze drying, 10 ml of PBS was added to all the cells and viability was measured to determine number of viable cells after freeze drying. All tests were done in triplicates. The results, shown in FIG. 8, showed that for all three bacteria, Na-γ-PGA was able to improve viability, compared to when no cryoprotectant was used. 10% Na-γ-PGA was a better cryoprotectant than 10% sucrose for L. casei, whereas its cryoprotective ability was comparable to 10% sucrose for both Bifidobacteria strains.

3.2. Na-γ-PGA as a Protectant in Fruit Juice and Simulated Gastric Juice

The effect of 2.5% Na-γ-PGA was tested on viability of the two Bifidobacteria strains when stored in orange juice for 39 days and simulated gastric juice for 4 h. To coat cells with Na-γ-PGA, bacteria were inoculated in TPY broth (22 h for B. breve and 16 h for B. longum) at 37° C. The culture was then centrifuged and washed with PBS to obtain cell pellets. Cells were then mixed thoroughly in a 10% Na-γ-PGA solution (1 gm Na-γ-PGA in 9 ml of deionised water). This mixture was frozen at −80° C. and freeze dried to obtain a dry powder with cells coated with Na-γ-PGA. 1 ml PBS was added to the dry cells and this solution was transferred to 40 ml of orange juice. Final concentration of Na-γ-PGA in fruit juice was ˜2.5%. For cells without γ-PGA coating, cells were inoculated in TPY broth under the aforementioned conditions. Cell pellets were obtained after centrifugation and washing with PBS. 1 ml of PBS was added to the cells. This solution was added to orange juice. Viability was measured at day 0, 2, 4, 6, 8, 11, 13, 20, 28 and 39 for cells with and without Na-γ-PGA coating on Bifidobacteria Selective Medium Agar (BSM agar). The results are shown in FIG. 9.

Cells that were not coated with Na-γ-PGA failed to survive in orange juice for 20 days. In contrast, cells with a Na-γ-PGA coating survived well for 39 days with a viability of about log 6-7 CFU/ml, when compared to their initial count of log 8-9 CFU/ml. Hence, Na-γ-PGA can be used to noticeably increase the shelf life of the food probiotic product.

Similarly the effect of 2.5% Na-γ-PGA was tested on viability of the 2 Bifidobacteria strains when stored in simulated gastric juice for 4 h. Simulated gastric juice with pH 2.0 was prepared in the lab to mimic the environment of the stomach. The aforementioned protocol was followed to coat cells with Na-γ-PGA. Viability was measured at 0 h, 1 h, 2 h, 3 h and 4 h for cells with and without Na-γ-PGA coating on BSM agar. The results are shown in FIG. 10. For cells that were coated with Na-γ-PGA, there was no noticeable loss in viability. However, cells without Na-γ-PGA coating died within 2 hours.

1. A modified probiotic microorganism, comprising a probiotic microorganism that is at least partially coated with a poly glutamic acid.

2. The modified probiotic microorganism of claim 1 wherein the poly glutamic acid is poly-γ-glutamic acid (γ-PGA).

3. The modified probiotic microorganism of claim 2 wherein the γ-PGA has a molecular weight of from 10,000 to 1,000,000.

4. (canceled)

5. (canceled)

6. The modified probiotic microorganism of claim 2 wherein the γ-PGA is produced in Bacillus subtilis or Bacillus licheniformis or Bacillus subtilis natto.

7. (canceled)

8. The modified probiotic microorganism of claim 1 wherein the probiotic microorganism is a bacterium.

9. An ingestible product comprising a plurality of modified probiotic microorganisms of claim 1.

10. The ingestible product of claim 9 wherein all or part, of some or all, of the plurality of modified probiotic microorganisms is coated with a polyglutamic acid.

11. (canceled)

12. The ingestible product of claim 9 wherein the ingestible product is milk based.

13. The ingestible product of claim 9 wherein the ingestible product is non-milk based.

14. A method of at least partially coating a probiotic microorganism with a poly glutamic acid comprising mixing the microorganism with a solution of a poly glutamic acid.

15. The method of claim 14 wherein the poly glutamic acid is provided in a concentration of from 2 to 15% (wv).

16. The method of claim 14 wherein the microorganism is provided in cell pellet form.

17. The method of claim 14 wherein the poly glutamic acid is poly-γ-glutamic acid (γ-PGA).

18. The method of claim 17 wherein the γ-PGA has a molecular weight of from 10,000 to 1,000,000.

19-22. (canceled)

23. The method of claim 14 wherein the probiotic microorganism is a bacterium.

24. A method of making poly-γ-glutamic acid (γ-PGA) comprising the steps of:
a) preparing a starter culture of a bacterial colony capable of producing γ-PGA in fermentation broth;
b) adding the starter culture to a growth medium;
c) producing γ-PGA in the growth medium.

25. The method of claim 24 wherein the bacterial colony is a colony of Bacillus subtilis or Bacillus licheniformis or Bacillus subtilis natto.

26. (canceled)

27. The method of claim 24 wherein the fermentation broth is tryptone soy broth (TSB).

28. The method of claim 24 wherein the growth media is growth media E or growth media GS.

29. The method of claim 24 wherein the bacterial colony is highly mucoid.

30-38. (canceled)
